(19) United States
(12) Patent Application Publication
(10) Pub. No.: US 2022/0080325 A1
(43) Pub. Date: Mar. 17, 2022

(54) ultra-lightweight thin stabilizing friction reducing video gaming vambrace

(71) Applicant: Dylan Drake, Sparta, WI (US)

(72) Inventor: Dylan Drake, Sparta, WI (US)

(21) Appl. No.: 17/021,333

(22) Filed: Sep. 15, 2020

(51) Int. Cl. A63F 13/98

(52) U.S. Cl. CPC A63F 13/98

(57) ABSTRACT

The present disclosure provides an ultra-lightweight bendable or heat moldable friction reducing, stabilizing wearable device worn on the underside wrist and forearm of a user, whereby the device provides 3.5 millimeters of clearance for the user's hand and decreases friction on a mouse pad surface or the like. The current application is about increasing stability and lowering the friction of garners under side wrists and forearms on support surfaces by applying an ultra-thin layer of polytetrafluoroethylene.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

Ultra-lightweight bendable and or moldable wearable forearm device for increasing performance, comfort and stability of a user of a computer mouse device or the like

### Technical Field

The present invention is a ultra-lightweight bendable and or moldable wearable forearm and wrist device apparatus for increasing performance and stability by reducing friction and improving comfort of a user of a computer mouse, or the like. The device may be used as a gaming vambrace, i.e. a vambrace that is intended to be worn on the wrist and forearm for use in video gaming in such a computer mouse is used for input.

### Description of the Related Art

Participants in a variety of activities that involve video game consoles, computer keyboards, and other input devices, often experience discomfort in and around the upper extremities, e.g. forearms and wrists. Many of these ailments can be directly attributed to improper positioning of the forearm and wrist, particularly when keyboarding, mouse use, or game console use which can cause overuse or repetitive-stress.

As is often the case with overuse or repetitive-stress injuries, minimizing the activity which caused the tendinitis is usually an effective treatment. However, patients are often unwilling to forego the offending activity for an extended period of time. Thus, there is a need for devices that help strengthen the musculature of the upper extremities and also relaxes and massages the fingers, wrists and forearms.

Many computer games are controlled at least partially by a computer mouse. Especially in so-called first-person-shooter games, the mouse may be used to control the aim of the player's weapon. Such control may require a large range of movements, from minute ones to relatively large ones.

Moreover, such movements often need to be performed very quickly, leading to a very high movement speed.

It is well known that use of computer mice may lead to injuries, such as carpal tunnel syndrome, and the like. Various devices have been produced with the aim to reduce the risks associated with the use of computer mice.

U.S. Pat. No. 5,925,007A discloses a carpal cuff, which is designed to reduce the risk of injuries on users of computer mice. Similar devices are disclosed in U.S. Pat. No. 6,082,684A, WO0236050A1 and WO09059492A1.

There is a need for further improvements of these devices.

None of the previous inventions and patents, taken either singly or in combination, is seen to describe the invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specifications, claims, drawings, and abstract as a whole.

The present invention relates to an ultra-lightweight stabilizing friction reducing bendable and or moldable underside forearm wrist apparatus device to increase performance and comfort in the use of a mouse while operating a computer or video gaming console.

The ultra-lightweight device comprises a wrist and forearm support with the underside adapted to bend and or mold to the curvature of the wrist and forearm and to reduce friction against a computer mouse pad surface or the like. While the upper side adapted to bend and or mold to the curvature of the underside of the users wrist and forearm for stabilization and comfort. Both of these layers combine to elevate the user's arm approximately 3.5 millimeters off of the support surface.

The underside adaptations for reducing friction against a computer mouse pad surface or the like shall be embodied as a thin bendable friction reducing polytetrafluoroethylene layer while the upper side adaptations for stability, comfort and to bend or mold with the curvature of the underside wrist and forearm shall be embodied as a thin ethylene-vinyl acetate layer attached together using plastic screw posts.

The ultra-lightweight thin device as described is attached to the user's wrist and forearm by use of two bilateral lightweight polypropylene webbing straps. These bilateral webbing straps go across the user's upper side forearm and wrist and fasten via method of velcro hook and loop arrangement.

The device may be referred to as a “vambrace” which implies a device that may be applied to the user's under side wrist and forearm.

This device is suitable for use in any video games that require precision, accuracy and speed as it reduces friction and provides a better pivot point on the support surface. Most commonly it will be used in first person shooter games such as Call of Duty®: Black Ops—Cold War, Call of Duty®: Modern Warfare, Counter-Strike: Global Offensive®, Fortnite®, Apex Legends®, Overwatch®, Valorant®, Battlefield 5®, PLAYERUNKNOWN'S BATTLEGROUNDS®, Rust®, Tom Clancy's Rainbow Six Siege®.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

FIG. 1 is a schematic perspective view of a gaming vambrace.

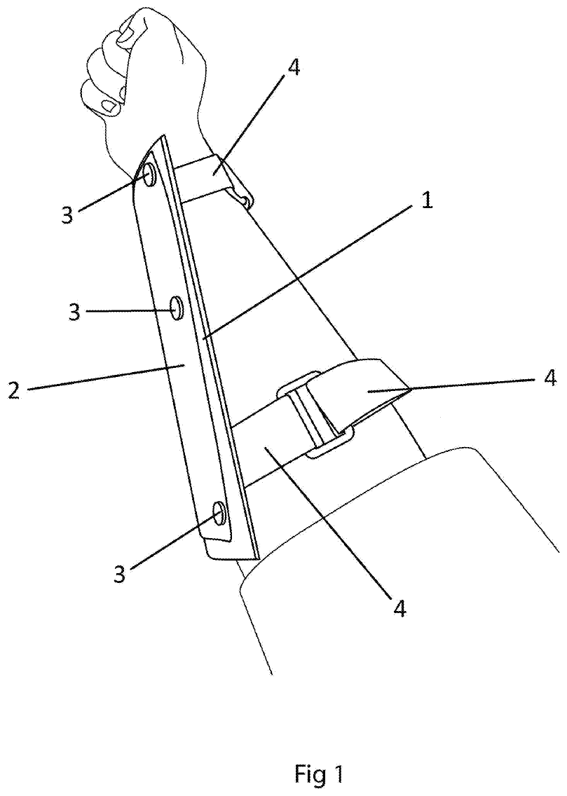

FIG. 2 is a schematic side view of the gaming vambrace.

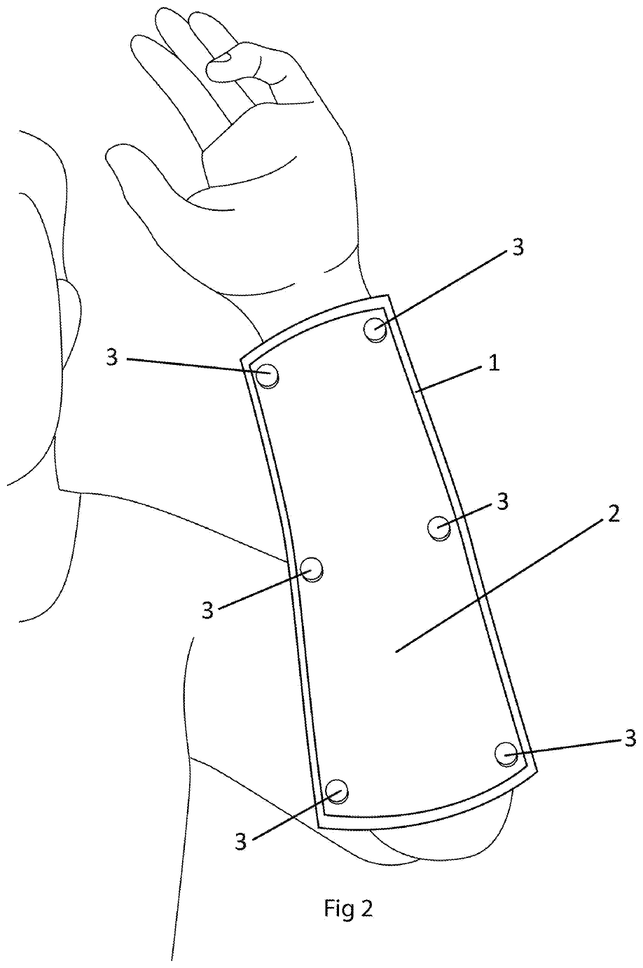

FIG. 3 schematically illustrates how a person can wear it on their wrist and forearm.

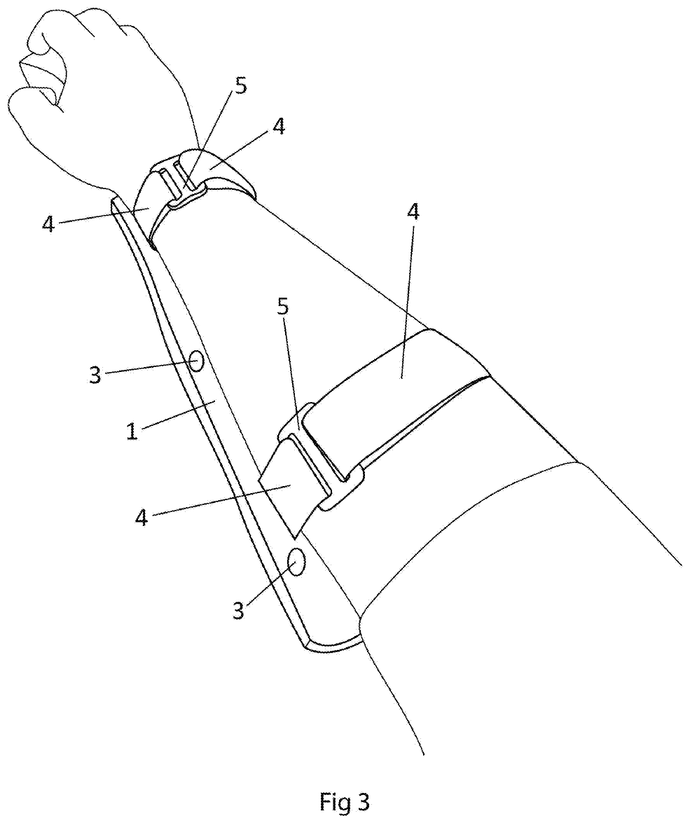

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

Referring to the drawings, a gaming vambrace will now be described. The gaming vambrace consists of a thin flexible and or heat moldable wrist and forearm support 1, connected to this base member is 2 the low friction layer attached together by 3, six screw posts with a diameter of 6.35 millimeters and head diameter of 9.525 millimeters.

The forearm and wrist support 1 may be formed as a bendable and or heat moldable and relatively curved, longitudinal member having a length that is from the start of the wrist to the beginning of the elbow.

The forearm and wrist support 1 may be formed of an ultra-lightweight 3.175-millimeter-thick Ethylene-vinyl acetate or the like layer that bends with the straps to allow it to mold to the users desired diameter. This layer of Ethylene-vinyl acetate may also be heat treated and molded to the user's exact wrist and forearm shape.

The low friction layer 2 on the underside of the forearm and wrist support 1 may be formed of a low friction layer such as polytetrafluoroethylene with a thickness of equal to or greater than 0.015 millimeters.

The forearm and wrist support 1 is connected to the user's wrist and forearm via two bilateral straps 4 made of one-inch polypropylene webbing fixed with a hook and loop Velcro arrangement and slider piece 5 to allow the user to attach it to their wrist and forearm with their preferred tension and diameter.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. An ultra-lightweight flexible and or heat moldable thin device for improving performance, comfort and stability of a user of a computer mouse or the like, consisting of;
a) 3.175-millimeter Ethylene-vinyl acetate
b) 0.381-millimeter Polytetrafluoroethylene
c) 25.4-millimeter Plastic Slider
d) 25.4-millimeter Lightweight Polypropylene Webbing
e) 19.05-millimeter Velcro hook and loop
f) 3.175-millimeter plastic screw post

2. An ultra-lightweight flexible and or heat moldable wearable thin device for improving performance, comfort and stability of a user of a computer mouse or the like, consisting of;
a) a wrist and forearm support 1 having an upper side molded and adapted to the curvature of the users under side wrist and forearm
b) friction reducing polytetrafluoroethylene underside of the device 2 consisting of removably attached and adapted for the curvature of the users underside wrist and forearm
c) elevating the users hand off of the support surface
c) the wrist and forearm, support 1 removably attached by means of screw posts to 2 combined together and attachable to the users wrist and forearm by means of straps 4
